(19) United States
(12) Patent Application Publication
(10) Pub. No.: US 2026/0325560 A1
(43) Pub. Date: Oct. 22, 2026

(54) COMBINING ANNAMYCIN WITH CLINICAL ANTICANCER AGENTS FOR INCREASED ACTIVITY

(71) Applicant: BOARD OF REGENTS, THE UNIVERSITY OF TEXAS SYSTEM, Austin, TX (US)

(72) Inventors: Waldemar PRIEBE, Houston, TX (US); Izabela FOKT, Houston, TX (US); Rafal Jerzy ZIELINSKI, Spring, TX (US); Stanislaw SKORA, Spring, TX (US); Roberto CARDENAS ZUNIGA, Houston, TX (US)

(73) Assignee: BOARD OF REGENTS, THE UNIVERSITY OF TEXAS SYSTEM, Austin, TX (US)

(21) Appl. No.: 19/651,159

(22) Filed: Apr. 17, 2026

Related U.S. Application Data
(60) Provisional application No. 63/791,478, filed on Apr. 19, 2025.

(51) Int. Cl. A61K 31/704; A61K 31/475; A61K 31/4995; A61K 31/635; A61K 31/675; A61K 31/7068; A61K 45/06; A61P 35/00; A61P 35/02; A61P 35/04

(52) U.S. Cl. CPC A61K 31/704; A61K 31/475; A61K 31/4995; A61K 31/635; A61K 31/675; A61K 31/7068; A61K 45/06; A61P 35/00; A61P 35/02; A61P 35/04

(57) ABSTRACT

In some aspects, disclosed herein are methods of treating cancer by administering to a patient a therapeutically effective amount of annamycin in combination with a therapeutically effective amount of a second anticancer compound. Also provided are pharmaceutical products comprising annamycin and cytarabine, azacitidine, venetoclax, vincristine, trabectedin, ifosfamide, irinotecan, gemcitabine, or a KRAS inhibitor, and methods of treating cancer using said pharmaceutical products.

### CROSS-REFERENCE TO RELATED APPLICATIONS

This application claims the benefit of priority to U.S. Provisional Application No. 63/791,478 filed Apr. 19, 2025, the entire contents of which are hereby incorporated by reference.

### I. Field of the Invention

The present invention relates generally to the field of chemistry, particularly medicinal chemistry. More particularly, it concerns therapies for the treatment of cancer, such as annamycin administered in combination with various anticancer compounds.

### II. Description of Related Art

Cancer is the leading cause of death in economically developed countries and the second leading cause of death in developing countries. Despite recent advances in chemotherapy and the development of agents targeted at the molecular level to interfere with the transduction and regulation of growth signals in cancer cells, the prognosis of patients with advanced cancer remains poor in general. Consequently, there is a persisting and urgent medical need to develop new therapies that can be added to existing treatments to increase survival without causing unacceptable toxicity.

Annamycin (ANN) is a potent topoisomerase II poison structurally related to doxorubicin (DOX) but displaying superior biological properties, such as high in vivo efficacy, reduced toxic side effects, and activity against cancers resistant to common therapeutic agents. ANN is currently clinically evaluated as a liposome formulated drug product, L-ANN. Importantly, L-ANN preclinical and clinical data clearly indicate a lack of cardiotoxic activity, which is a common dose-limiting factor in this class of therapeutics. In addition, ANN and L-ANN display unique organotropism that significantly differs from that of DOX. This finding further supported the synergistic or additive effects of combining ANN with different anticancer agents. The efficacy of L-ANN has been established in multiple tumor models including leukemia and solid tumors. Thus, a need exists for combination therapies comprising ANN and other compounds for the treatment of cancer.

### SUMMARY

The present disclosure provides methods and pharmaceutical products for the treatment and prevention of cancer. In some aspects, the present disclosure provides a method of treating a cancer, the method comprising administering to a patient in need thereof a therapeutically effective amount of annamycin or a pharmaceutically acceptable liposomal formulation or prodrug thereof in combination with a therapeutically effective amount of a second anticancer compound or a pharmaceutically acceptable salt or liposomal formulation, or prodrug thereof.

In some embodiments, the second anticancer compound is an alkylating agent. In some embodiments, the second anticancer compound is a methylating agent. In some embodiments, the second anticancer compound is cytarabine, azacitidine, venetoclax, vincristine, trabectedin, ifosfamide, cyclophosphamide, irinotecan, or gemcitabine. In some embodiments, the second anticancer compound is a KRAS inhibitor. In some embodiments, the second anticancer compound is a pan-KRAS inhibitor. In other embodiments, the second anticancer compound is a selective KRAS inhibitor.

In some aspects, the therapeutic effect in the patient is greater than the aggregate achieved by either annamycin or the second anticancer compound. In some embodiments, the cancer is metastatic. In some embodiments, the cancer is drug-resistant.

In some embodiments, the cancer is selected from the group consisting of mesothelioma, bladder cancer, lung cancer, pancreatic cancer, skin cancer, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma, cancer of the urethra, cancer of the penis, testicular cancer, hepatocellular (hepatic and/or biliary duct) cancer, primary or secondary central nervous system tumor, primary or secondary brain tumor, osteosarcoma, Hodgkin's disease, chronic or acute leukemia, acute myeloid leukemia, acute lymphocytic leukemia (ALL), chronic myeloid leukemia, lymphocytic lymphoma, lymphoblastic leukemia, follicular lymphoma, lymphoid malignancies of T-cell or B-cell origin, melanoma, multiple myeloma, oral cancer, prostate cancer, cancer of the kidney and/or meter, renal cell carcinoma, carcinoma of the renal pelvis, neoplasm of the central nervous system, primary central nervous system lymphoma, non-Hodgkin's lymphoma, spinal axis tumor, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In certain embodiments, the cancer is sarcoma. In other embodiments, the cancer is pancreatic cancer, such as pancreatic ductal adenocarcinoma. In still other embodiments, the cancer is acute myeloid leukemia or acute lymphocytic leukemia. In yet other embodiments, the cancer is lung cancer, such as lung metastasis of fibrosarcoma origin. In other embodiments, the cancer is osteosarcoma.

In some embodiments, the patient is a mammal. In some embodiments, the patient is a human. In some embodiments, annamycin is administered concurrently with the second anticancer compound. In some embodiments, annamycin is administered sequentially with the second anticancer compound. In some embodiments, annamycin and the second anticancer compound are co-formulated. In some embodiments, annamycin and the second anticancer compound are co-formulated in a pharmaceutical composition comprising a pharmaceutically acceptable excipient. In some embodiments, annamycin and the second anticancer compound are formulated in separate dosage forms.

In some embodiments, the method further comprises administering a pharmaceutically effective amount of at least one immunotherapeutic agent to the patient. In some embodiments, the method further comprises administering radiation therapy to the patient. In some embodiments, treating a cancer in a patient in need thereof comprises one of more of the following:
(a) increasing survival time of the patient;
(b) reducing volume of the primary cancer;
(c) retarding growth of the primary cancer;
(d) reducing the number of metastatic tumors;
(e) reducing the volume of metastatic tumors; and
(f) retarding growth of metastatic tumors.

In some embodiments, wherein the cancer is a solid tumor and the treatment results in a reduction in tumor size of at least 15%. In certain embodiments, the reduction in tumor size is at least 30%. In some embodiments, treating cancer does not result in an annamycin-induced toxicity of such severity that repeated administration of annamycin is contraindicated. In some embodiments, the annamycin-induced toxicity is cardiotoxicity.

In some aspects, the present disclosure provides a pharmaceutical product comprising:
(a) a first composition comprising annamycin; and
(b) a second composition comprising cytarabine, azacitidine, venetoclax, vincristine, trabectedin, ifosfamide, cyclophosphamide, irinotecan, gemcitabine, or a KRAS inhibitor.

In some embodiments, the first composition further comprises a pharmaceutically acceptable excipient. In some embodiments, the second composition further comprises a pharmaceutically acceptable excipient. In some embodiments, the pharmaceutical product is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, the pharmaceutical product is formulated for oral administration. In some embodiments, the pharmaceutical product is formulated for administration via injection.

In some aspects, the present disclosure provides a method of treating cancer in a patient in need thereof, comprising administering to the patient pharmaceutical product described herein.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description.

FIGS. 1A-1C show the efficacy of L-ANN and IFO in experimental lung metastatic model of fibrosarcoma. Female C57B6 mice were injected intravenously with 1×10⁵ mouse MCA205 fibrosarcoma cells. On day 10 animals were randomized into four experimental groups (n=8-9) receiving (i) vehicle (n=8), L-ANN at 4 mg/kg (n=8), IFO at 90 mg/kg (n=8) and combination of both (n=9). Arrows indicate injection of the drug. MS-Median Survival.

FIGS. 2A-2E show the efficacy of L-ANN and Irinotecan in orthotopic MDA PATC53 tumor model. Female nude athymic mice were surgically implanted with 1 million of human MDA PATC53 Luc PDAC cells (intrapancreatic tumor injection). On the day 8th, the animals were randomized into four experimental groups receiving i) vehicle, ii) irinotecan (IRINO), 5 mg/kg, iii) L-ANN 4 mg/kg (n=10) and iv) combination of both (n=9). The rate of tumor growth was monitored using bioluminescent imaging (BLI), 10 minutes after subcutaneous administration of D-Luciferin at 75 mg/kg. Combination benefits were assessed using Kaplan-Mayer survival analysis and compared between the vehicle- and single-agent treated groups. Survival benefits were analyzed using long-rank (Mantel-Cox) test in GraphPad Prism software. The distribution of BL signals was analyzed using noparametric one-way ANOVA (Kruskal-Wallis test)

FIGS. 3A-3B show the efficacy of L-ANN and GEM in orthotopic MDA PATC53 tumor model. Female nude athymic mice were orthotopically implanted with 1.0 million of human MDA PATC53 PDAC cells in mixture with Matrigel (50%, 50 μl). On day 8, the animals were randomized into four experimental groups (n=10) receiving i) vehicle, ii) gemcitabine 50 mg/kg, iii) L-ANN at 2 mg/kg and iv) combination of gemcitabine and L-ANN. Both agents were administered on a once-a-week schedule for six cycles. Survival benefits were analyzed using long-rank (Mantel-Cox) test in GraphPad Prism software. The distribution of BL signals was analyzed using nonparametric one-way ANOVA (Kruskal-Wallis test).

FIG. 4 shows the efficacy of L-ANN and VCR in murine acute myeloid leukemia model. Female C57B6 albino mice received bolus, IV injection of AML-Turq-2 cells. Day five following tumor inoculation, the mice were randomized into four experimental groups (n=5) receiving i) vehicle, ii) L-ANN 4 mg/kg, iii) VCR 1 mg/kg and iv) combination of L-ANN and VCR. Arrows indicate the administration of L_ANN and/or VCR Survival benefits were analyzed using long-rank (Mantel-Cox) test in GraphPad Prism software.

FIGS. 5A-5E show the efficacy of L-ANN and IFO in human osteosarcoma model. Human osteosarcoma cells (143B; 1 million/mouse) were injected subcutaneously into female athymic mice. On day ten when the average tumor volume reached ~100 mm³, the mice were randomized into four groups (n=10) receiving i) vehicle, ii) IFO 90 mg/kg, L-ANN 3 mg/kg and iv) combination of both. L-ANN was administered once a week and IFO was given on a three-times-a-week schedule (Mon-Wed-Fri). The tumor growth was monitored based on tumor volume, estimated using ellipsoid equation based on tumor diameter measurement. FIG. 5B, FIG. 5C and FIG. 5E show the signal distribution at the study termination on day 30. Combination index (CI) was calculated as follows: CI=((cA+cB)−cA×cB )/cAB where cA represents the inhibitory rate of compound A, cB represents the inhibitory rate of compound B, and cAB represents the inhibitory rate of combination treatment of compound A and B. Synergy is defined as CI lower than 1.0, and antagonism is defined as CI significantly greater than 1.0.

FIGS. 6A-6C show an in vitro analysis of synergy between ANN and Trabectedin in MPanc 96 cells. ATCC available Mpanc-96 cells were plated in 96 well plate at 3×10∧3 cells/well and incubated overnight at 5% CO₂ at 37° C. The next day, the cells were exposed to increasing concentration of ANN and Trabectedin in stable ratio 50:1 and constant vehicle (0.5% DMSO). After 72 hours, the viability of the cells was assessed using Resazurin assay. Viability was normalized to vehicle and “no cell”-treated control, and expressed as percentage, The viability matrix was analyzed using Bliss algorithm in Combenefit Software to identify potential region of synergy.

FIGS. 7A-7C show an in vitro analysis of synergy between ANN and Trabectedin MiaPaCa-2 cells. ATCC available MiaPaCa-2 cells were plated in 96 well plate at 3×10∧3 cells/well and incubated overnight at 5% CO₂ at 37C. The next day, the cells were exposed to increasing concentration of ANN and Trabectedin in stable ratio 50:1 and constant vehicle (0.5% DMSO). After 72 hours, the viability of the cells was assessed using Resazurin assay. Viability was normalized to vehicle and “no cell”-treated control, and expressed as percentage, The viability matrix was analyzed using Bliss algorithm in Combenefit Software to identify potential region of synergy.

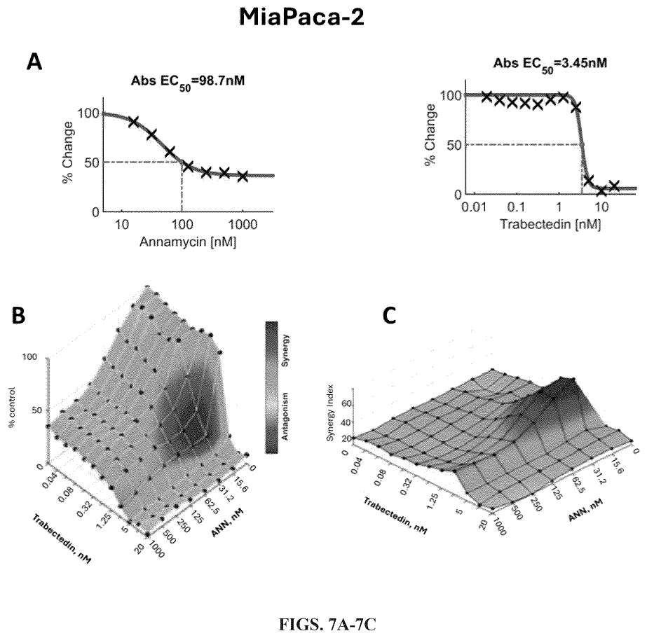

FIGS. 8A-8B show an in vitro analysis of synergy between ANN and Trabectedin in a panel of Sarcoma cell lines. Established murine sarcoma cell lines: K7M3 (osteosarcoma), MCA205 (fibrosarcoma) and human osteosarcoma cells (143B) were plated at 500 cells/well on 384 well plate. After overnight attachment, the cells were treated with a combination of TRB, and ANN at fixed ratio of 1:80; 1:40; 1:20; and 1:10. The viability of the cells following 72 h exposure, was assessed using CellTiterGlo assay. The data was normalized to vehicle-treated control and no-cell control and expressed as fraction control viability. The obtained matrices were analyzed using CaluSyn Version 2.11 to compute the combination indexes (CIs). CI below 1 indicates synergistic nature of interaction, 1.0—additive and CI above 1.0 points to antagonism between tested agents. CI_{ED50}, CI_{ED75}, CI_{ED90,}-combinatory index calculated of fraction effect ED50, ED75, ED90. R—linear correlation coefficient.

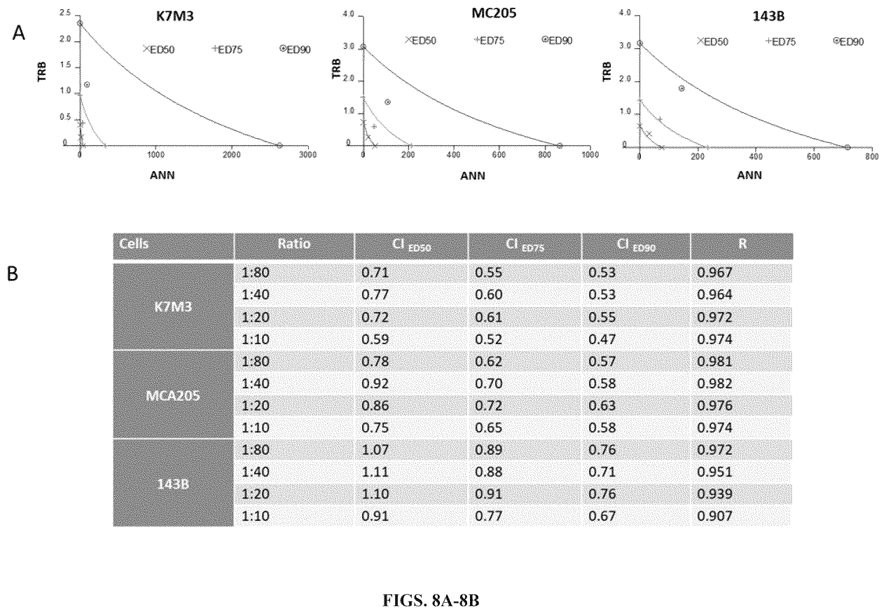

FIGS. 9A-9B illustrate the efficacy of L-ANN and Cytarabine (ARA-C) in syngeneic AML model. Female C57B6 mice were injected intravenously with 1×10⁵ AML-Turq2 cells. On day five animals were randomized into four experimental groups (n=10−12) receiving: vehicle, L-ANN, ARA-C and L-ANN plus ARA-C. On day 36 of the study distribution of leukemic blasts in selected animals was analyzed using ex vivo confocal microscopy. MS—Median Survival.

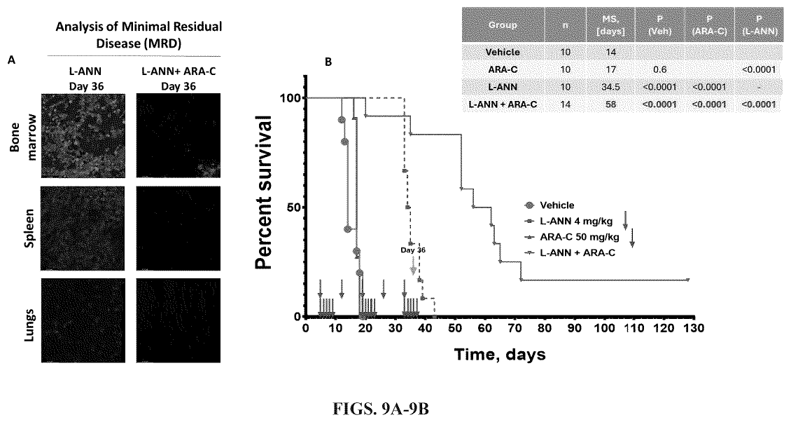

FIGS. 10A-10B show in vitro analysis of synergy between ANN and Azacitidine in MOLM-13 human myeloid leukemia model. MOLM-13 cells were plated at 3500 cells/well on 96-well plates. The cells were treated with a combination of AZA, and ANN at fixed ratio of 1:400, 1:200, 1:100, and 1:50 (ANN to AZA). The viability of the cells following 72 h drug exposure, was assessed using Resazurin assay. The data was normalized to vehicle-treated control and no-cell control and expressed as fraction of control viability. The obtained matrices were analyzed using CaluSyn Version 2.11 to compute the combination indexes (CIs). CI below 1 indicates synergistic nature of interaction, 1.0-additive effect and CI above 1.0 indicates antagonism between tested agents. CI_{ED50}, CI_{ED75}, CI_{ED90,}-combinatory index calculated of fraction effect ED50, ED75, ED90. R—linear correlation coefficient.

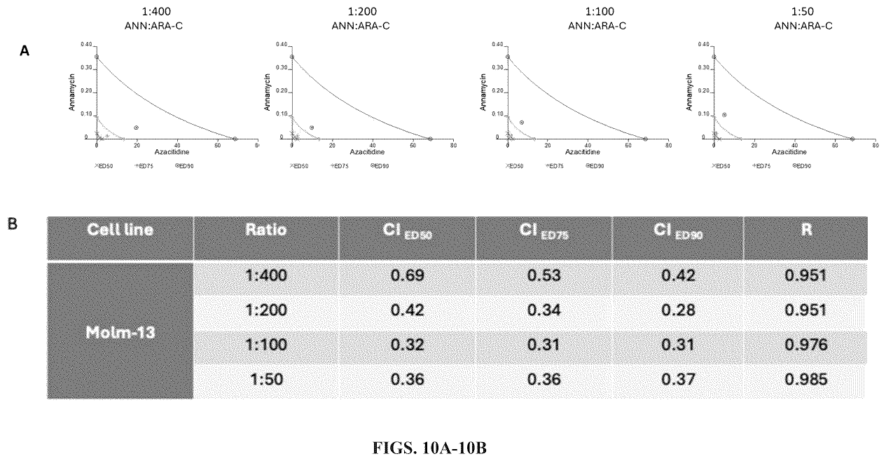

### DESCRIPTION OF ILLUSTRATIVE EMBODIMENTS

Disclosed herein are combination therapies comprising annamycin, which may be useful for the treatment of cancer. In one aspect, a method of treating cancer is provided, the method comprising administering to a patient in need thereof a therapeutically effective amount of annamycin or a pharmaceutically acceptable liposomal formulation or prodrug thereof in combination with a therapeutically effective amount of a second anticancer compound or a pharmaceutically acceptable salt or liposomal formulation, or prodrug thereof. In one aspect, a pharmaceutical product is provided comprising: (a) a first composition comprising annamycin; and (b) a second composition comprising cytarabine, azacitidine, venetoclax, vincristine, trabectedin, ifosfamide, cyclophosphamide, irinotecan, or gemcitabine. In one aspect, a pharmaceutical product is provided comprising: (a) a first composition comprising annamycin; and (b) a second composition comprising a KRAS inhibitor. In one aspect, a method of treating cancer in a patient in need thereof is provided, comprising administering to the patient pharmaceutical product described herein.

In some aspects, the methods and pharmaceutical products described in the sections that follow, taken together or individually, are advantageous for the treatment of cancers. In some aspects, the methods and pharmaceutical products described in the sections that follow, taken together or individually, may result in reduced toxicity. In some aspects, the methods and pharmaceutical products described in the sections that follow, taken together or individually, may result in reduced cardiotoxicity. In particular, the methods and pharmaceutical products described in the sections that follow, taken together or individually, may result in reduced cardiotoxicity in comparison to known combination therapies comprising administration of doxorubicin and a second therapeutic agent, including but not limited to combination therapies comprising administration of doxorubicin and ifosfamide. In some aspects, the methods and pharmaceutical products described in the sections that follow, taken together or individually, may result in improved uptake of the first composition and/or the second composition. In some aspects, the methods and pharmaceutical products described in the sections that follow, taken together or individually, may result in reduced toxicity. In some aspects, the methods and pharmaceutical products described in the sections that follow, taken together or individually, may result in synergistic or additive therapeutic effects for the treatment of cancer.

The efficacy of annamycin in combination with different anticancer agents in order to identify the potential clinical application of annamycin alone and with a therapeutic partner. The activity of annamycin in combination with selected FDA approved drugs was assessed in vitro after 72 hours of treatment using a cytotoxicity assay followed by determination of the combination indexes. In separate in vitro experiments, annamycin activity was tested against drug resistant cell lines, including cells resistant to cytarabine and venetoclax. The most efficacious drug combinations from the in vitro studies were then tested using in vivo models of leukemia and solid tumors, including sarcoma and pancreatic cancer.

The in vitro screening performed in set of cancer cell lines led to selection of the following agents combined with annamycin for testing efficacy in clinically relevant animal models: 1) Cytarabine 2) Azacitidine 3) Venetoclax, 4) Vincristine in AML model; 5) Ifosfamide in fibrosarcoma; 6) Irinotecan and 7) Gemcitabine in PATC53 pancreatic cancer model. All combinations listed above showed improved survival when compared to single agent control groups. Importantly, preclinical studies led to the clinical development of L-annamycin plus cytarabine regimen, a combination that demonstrated clinical anticancer activity in Phase I/II studies in AML patients. Annamycin is a versatile drug forming efficacious combinations with numerous mechanistically different anticancer agents.

The above examples of favorable attributes listed above are for illustration purposes and should not be construed to limit the observed or contemplated benefits associated with the methods disclosed herein. Any of the individual compositions or methods disclosed in the present application may exhibit any combination of the benefits listed above. As mentioned above, the methods disclosed herein may also have one or more other attributes deemed favourable to or by the practitioner, optionally in combination with any of the attributes listed in the paragraphs above. These examples of the advantageous attributes should not be construed to limit in any way the possible advantages of the presently disclosed methods.

The technical solutions in the embodiments of the present application will be clearly and completely described below with reference to the accompanying figures in the embodiments of the present application. Based on the embodiments in the present application, all other embodiments obtained by those skilled in the art without creative effort fall into the protection scope of the present application.

### III. Compounds and Compositions

The compounds and compositions of the present invention (also referred to as “compounds and compositions of the present disclosure”) are described, for example in the summary of the invention section, in the examples section, and in the claims. The compounds can be further modified and optimized using the principles and techniques of organic chemistry. Such principles and techniques are taught, for example, in Smith, March's Advanced Organic Chemistry: Reactions, Mechanisms, and Structure, (2013), which is incorporated by reference herein. In addition, the compounds and compositions may be further modified and optimized for preparative, pilot-or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds and compositions of the present invention may in some embodiments be combined to form a therapy and/or pharmaceutical product advantageous for the prevention and treatment of one or more diseases or disorders discussed herein or otherwise. In certain embodiments, the disease or disorder is a cancer.

All the compounds and compositions of the present invention may in some embodiments be used for the prevention and treatment of one or more diseases or disorders discussed herein or otherwise. In some embodiments, one or more of the compounds characterized or exemplified herein as a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds and compositions of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In some embodiments, compounds of the present invention function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present invention exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### IV. Pharmaceutical Formulations And Routes Of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal Dose (mg/kg)×(Animal Kₘ/Human Kₘ)

Use of the Km factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount is from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

In some aspects, the compounds of the present disclosure are formulated to create a pharmaceutical product comprising: (a) a first composition comprising annamycin; and (b) a second composition comprising cytarabine, azacitidine, venetoclax, vincristine, trabectedin, ifosfamide, cyclophosphamide, irinotecan, or gemcitabine. In one aspect, the compounds of the present disclosure are formulated to create a pharmaceutical product comprising: (a) a first composition comprising annamycin; and (b) a second composition comprising a KRAS inhibitor. In some aspects, the pharmaceutical product can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of pharmaceutical product will be dependent on the subject being treated, the severity and type of the affliction, and the manner of administration. The exact amount of pharmaceutical product is readily determined by one of skill in the art based on the age, weight, sex, and physiological condition of the subject. Effective doses can be extrapolated from dose-response curves derived from in vitro or animal model test systems.

Upon formulation, the pharmaceutical product will be administered in a manner compatible with the dosage formulation and in such amount as is therapeutically effective.

The pharmaceutical product can be administered to the subject using any method known to those of ordinary skill in the art. For example, a pharmaceutically effective amount of a composition comprising nanoparticles may be administered intravenously, intracerebrally, intracranially, intrathecally, into the substantia nigra or the region of the substantia nigra, intradermally, intraarterially, intraperitoneally, intralesionally, intratracheally, intranasally, topically, intramuscularly, intraperitoneally, subcutaneously, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990).

An effective amount of the pharmaceutical product is determined based on the intended goal, for example inhibition of cell death. The quantity to be administered, both according to number of treatments and dose, depends on the subject to be treated, the state of the subject, the protection desired, and the route of administration. Precise amounts of the therapeutic agent also depend on the judgment of the practitioner and are peculiar to each individual.

For example, a dose of the therapeutic agent may be about 0.0001 milligrams to about 1.0 milligrams, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligrams. In even more particular embodiments, a dose may be at least 10 milligrams. In further embodiments, a dose is at least 100 milligrams or higher.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The dose can be repeated as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

In certain embodiments, the method may provide a continuous supply of the pharmaceutical product to the patient. This could be accomplished by catheterization, followed by continuous administration of the therapeutic agent. The administration could be intra-operative or post-operative.

### V. Definitions

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses. The patient may be a “cancer patient”, i.e. one who is suffering or at risk for suffering from one or more symptoms of cancer. A “subject” or an “individual” for purposes of treatment refers to any animal classified as a mammal, including humans, domestic and farm animals, and zoo, sports, or pet animals, such as dogs, horses, cats, cows, etc. Preferably, the mammal is human.

The term “mammal” includes, but is not limited to, humans, mice, rats, guinea pigs, monkeys, dogs, cats, horses, cows, pigs, sheep, and poultry. In one embodiment, the mammal is a human male or a human female.

A “population” of patients refers to a group of patients with cancer, such as in a clinical trial, or as seen by oncologists following FDA approval for a particular medication, such as unresectable or metastatic melanoma cancer therapy.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono-and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2″, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “a method of treating” or its equivalent, when applied to, for example, cancer refers to a procedure or course of action that is designed to reduce or eliminate the number of cancer cells in a patient, or to alleviate the symptoms of a cancer. “A method of treating cancer or another proliferative disorder does not necessarily mean that the cancer cells or other disorder will, in fact, be eliminated, that the number of cells or disorder will, in fact, be reduced, or that the symptoms of a cancer or other disorder will, in fact he alleviated. Often, a method of treating cancer will be performed even with a low likelihood of success, but which, given the medical history and estimated survival expectancy of a patient is nevertheless deemed to induce an overall beneficial course of action.

The terms “co-administration” or “coadministering” refer to the administration of annamycin and said anticancer compound as two separate formulations or within one single formulation. The co-administration can be simultaneous or sequential in either order. In one further embodiment, there is a time period while both (or all) active agents simultaneously exert their biological activities. Annamycin and the anticancer compound are co-administered either simultaneously or sequentially (e.g. via an intravenous (i.v.) through a continuous infusion (one for annamycin and eventually one for the anticancer compound; or the anticancer compound is administered orally). When both therapeutic agents are co-administered sequentially, the agents are administered in two separate administrations that are separated by a “specific period of time”. The term specific period of time is meant anywhere from 1 hour to 15 days. For example, one of the agents can be administered within about 15, 14, 13, 12, 11, 10, 9, 8, 7, 6, 5, 4, 3, 2, or 1 day, or 24, 23, 22, 21, 20, 19, 18, 17, 16, 15, 14, 13, 12, 11, 10, 9, 8, 7, 6, 5, 4, 3, 2 or 1 hour from the administration of the other agent, and, in one embodiment, the specific period time is 10, 9, 8, 7, 6, 5, 4, 3, 2, or 1 day, or 24, 23, 22, 21, 20, 19, 18, 17, 16, 15, 14, 13, 12, 11, 10, 9, 8, 7, 6, 5, 4, 3, 2 or 1 hour.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a tumor.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell growth. Examples of cancer include but are not limited to, mesothelioma, bladder cancer, lung cancer, pancreatic cancer, skin cancer, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma, cancer of the urethra, cancer of the penis, testicular cancer, hepatocellular (hepatic and/or biliary duct) cancer, primary or secondary central nervous system tumor, primary or secondary brain tumor, osteosarcoma, Hodgkin's disease, chronic or acute leukemia, acute myeloid leukemia, acute lymphocytic leukemia (ALL), chronic myeloid leukemia, lymphocytic lymphoma, lymphoblastic leukemia, follicular lymphoma, lymphoid malignancies of T-cell or B-cell origin, melanoma, multiple myeloma, oral cancer, prostate cancer, cancer of the kidney and/or meter, renal cell carcinoma, carcinoma of the renal pelvis, neoplasm of the central nervous system, primary central nervous system lymphoma, non-Hodgkin's lymphoma, spinal axis tumor, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. Indications

Provided herein are methods for treating or delaying progression of cancer, in an individual comprising administering to the individual an effective amount of a pharmaceutical product comprising annamycin. In some embodiments, there is provided a method for inhibiting growth and/or survival of metastatic cancer cells, comprising treating the subject with a therapeutically effective amount of a pharmaceutical product comprising annamycin. The cancer may be a solid tumor, metastatic cancer, or non-metastatic cancer. In certain embodiments, the cancer may originate in the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In certain embodiments, the cancer is human ovarian cancer or breast cancer. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present disclosure may also be used to treat a non-cancerous disease (e.g., a fungal infection, a bacterial infection, a viral infection, and/or a neurodegenerative disease).

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

Administration may be by injection, for example, intravenous, intrathecal, intramuscular or subcutaneously, intranasal, and oral. Combinations of treatment that provide systemic availability of the therapeutic may also be used. The therapeutically effective amount of the pharmaceutical product is that amount that achieves a desired effect in a subject being treated. For instance, this can be the amount of pharmaceutical product necessary to inhibit advancement, or to cause regression of cancer, or which is capable of relieving symptoms caused by a cancer.

In some embodiments, the method of the present invention involves the administration to a mammal e.g., a human patient in need of such a therapy a therapeutically effective amount of annamycin or a pharmaceutically acceptable liposomal formulation or prodrug thereof and an anticancer compound or a pharmaceutically acceptable salt or liposomal formulation or prodrug thereof. In preferred embodiments, the anticancer compound is cytarabine, azacitidine, venetoclax, vincristine, trabectedin, ifosfamide, cyclophosphamide, irinotecan, gemcitabine, or a pharmaceutically acceptable salt or liposomal formulation or prodrug thereof. In preferred embodiments, the anticancer compound is a KRAS inhibitor, or a pharmaceutically acceptable salt or liposomal formulation or prodrug thereof. In another embodiment, a pharmaceutical product of the invention is administered in a therapeutically effective amount to a subject in need thereof to treat a disease of abnormal cell growth and/or dysregulated apoptosis.

Examples of such diseases include, but are not limited to: mesothelioma, bladder cancer, lung cancer, pancreatic cancer, skin cancer, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma, cancer of the urethra, cancer of the penis, testicular cancer, hepatocellular (hepatic and/or biliary duct) cancer, primary or secondary central nervous system tumor, primary or secondary brain tumor, osteosarcoma, Hodgkin's disease, chronic or acute leukemia, acute myeloid leukemia, acute lymphocytic leukemia (ALL), chronic myeloid leukemia, lymphocytic lymphoma, lymphoblastic leukemia, follicular lymphoma, lymphoid malignancies of T-cell or B-cell origin, melanoma, multiple myeloma, oral cancer, prostate cancer, cancer of the kidney and/or meter, renal cell carcinoma, carcinoma of the renal pelvis, neoplasm of the central nervous system, primary central nervous system lymphoma, non-Hodgkin's lymphoma, spinal axis tumor, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In more particular embodiments, the method of the present invention involves the administration to a mammal, e.g., a human patient, in need of such a therapy a therapeutically effective amount of annamycin or a pharmaceutically acceptable liposomal formulation or prodrug thereof and an anticancer compound, such as cytarabine, azacitidine, venetoclax, vincristine, trabectedin, ifosfamide, cyclophosphamide, irinotecan, gemcitabine, or a pharmaceutically acceptable salt or liposomal formulation or prodrug thereof to treat sarcoma, pancreatic cancer, such as pancreatic ductal adenocarcinoma, acute myeloid leukemia, acute lymphocytic leukemia, lung cancer, such as lung metastasis of fibrosarcoma origin, or osteosarcoma. In more particular embodiments, the method of the present invention involves the administration to a mammal, e.g., a human patient, in need of such a therapy a therapeutically effective amount of annamycin or a pharmaceutically acceptable liposomal formulation or prodrug thereof and an anticancer compound, such as a KRAS inhibitor, or a pharmaceutically acceptable salt or liposomal formulation or prodrug thereof to treat sarcoma, pancreatic cancer, such as pancreatic ductal adenocarcinoma, acute myeloid leukemia, acute lymphocytic leukemia, lung cancer, such as lung metastasis of fibrosarcoma origin, colorectal cancer, or osteosarcoma.

In one embodiment, the method of the present involves the administration to a mammal, e.g., a human patient in need of such a therapy, a therapeutically effective amount of annamycin or a pharmaceutically acceptable liposomal formulation or prodrug thereof and an anticancer compound. In one embodiment, the anticancer compound is cytarabine. In a particular embodiment, the method of the present invention involves the administration to a mammal, e.g., a human patient, in need of such a therapy a therapeutically effective amount of annamycin or a pharmaceutically acceptable liposomal formulation or prodrug thereof and a therapeutically effective amount of cytarabine or a pharmaceutically acceptable salt or liposomal formulation thereof to treat diseases including but not limited to: mesothelioma, bladder cancer, lung cancer, pancreatic cancer, skin cancer, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma, cancer of the urethra, cancer of the penis, testicular cancer, hepatocellular (hepatic and/or biliary duct) cancer, primary or secondary central nervous system tumor, primary or secondary brain tumor, osteosarcoma, Hodgkin's disease, chronic or acute leukemia, acute myeloid leukemia, acute lymphocytic leukemia (ALL), chronic myeloid leukemia, lymphocytic lymphoma, lymphoblastic leukemia, follicular lymphoma, lymphoid malignancies of T-cell or B-cell origin, melanoma, multiple myeloma, oral cancer, prostate cancer, cancer of the kidney and/or meter, renal cell carcinoma, carcinoma of the renal pelvis, neoplasm of the central nervous system, primary central nervous system lymphoma, non-Hodgkin's lymphoma, spinal axis tumor, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In one embodiment, the method of the present involves the administration to a mammal, e.g., a human patient in need of such a therapy, a therapeutically effective amount of annamycin or a pharmaceutically acceptable liposomal formulation or prodrug thereof and an anticancer compound. In one embodiment, the anticancer compound is azacitidine. In a particular embodiment, the method of the present invention involves the administration to a mammal, e.g., a human patient, in need of such a therapy a therapeutically effective amount of annamycin or a pharmaceutically acceptable liposomal formulation or prodrug thereof and a therapeutically effective amount of azacitidine or a pharmaceutically acceptable salt or liposomal formulation thereof to treat diseases including but not limited to: mesothelioma, bladder cancer, lung cancer, pancreatic cancer, skin cancer, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma, cancer of the urethra, cancer of the penis, testicular cancer, hepatocellular (hepatic and/or biliary duct) cancer, primary or secondary central nervous system tumor, primary or secondary brain tumor, osteosarcoma, Hodgkin's disease, chronic or acute leukemia, acute myeloid leukemia, acute lymphocytic leukemia (ALL), chronic myeloid leukemia, lymphocytic lymphoma, lymphoblastic leukemia, follicular lymphoma, lymphoid malignancies of T-cell or B-cell origin, melanoma, multiple myeloma, oral cancer, prostate cancer, cancer of the kidney and/or meter, renal cell carcinoma, carcinoma of the renal pelvis, neoplasm of the central nervous system, primary central nervous system lymphoma, non-Hodgkin's lymphoma, spinal axis tumor, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In one embodiment, the method of the present involves the administration to a mammal, e.g., a human patient in need of such a therapy, a therapeutically effective amount of annamycin or a pharmaceutically acceptable liposomal formulation or prodrug thereof and an anticancer compound. In one embodiment, the anticancer compound is venetoclax. In a particular embodiment, the method of the present invention involves the administration to a mammal, e.g., a human patient, in need of such a therapy a therapeutically effective amount of annamycin or a pharmaceutically acceptable liposomal formulation or prodrug thereof and a therapeutically effective amount of venetoclax or a pharmaceutically acceptable salt or liposomal formulation thereof to treat diseases including but not limited to: mesothelioma, bladder cancer, lung cancer, pancreatic cancer, skin cancer, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma, cancer of the urethra, cancer of the penis, testicular cancer, hepatocellular (hepatic and/or biliary duct) cancer, primary or secondary central nervous system tumor, primary or secondary brain tumor, osteosarcoma, Hodgkin's disease, chronic or acute leukemia, acute myeloid leukemia, acute lymphocytic leukemia (ALL), chronic myeloid leukemia, lymphocytic lymphoma, lymphoblastic leukemia, follicular lymphoma, lymphoid malignancies of T-cell or B-cell origin, melanoma, multiple myeloma, oral cancer, prostate cancer, cancer of the kidney and/or meter, renal cell carcinoma, carcinoma of the renal pelvis, neoplasm of the central nervous system, primary central nervous system lymphoma, non-Hodgkin's lymphoma, spinal axis tumor, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In one embodiment, the method of the present involves the administration to a mammal, e.g., a human patient in need of such a therapy, a therapeutically effective amount of annamycin or a pharmaceutically acceptable liposomal formulation or prodrug thereof and an anticancer compound. In one embodiment, the anticancer compound is vincristine. In a particular embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of vincristine or a pharmaceutically acceptable salt or liposomal formulation thereof to treat acute myeloid leukemia or acute lymphocytic leukemia. In one embodiment, the method of the present invention involves the administration to a mammal, e.g., a human patient, in need of such a therapy a therapeutically effective amount of annamycin or a pharmaceutically acceptable liposomal formulation or prodrug thereof and a therapeutically effective amount of vincristine or a pharmaceutically acceptable salt or liposomal formulation thereof to treat acute myeloid leukemia.

In one embodiment, the method of the present involves the administration to a mammal, e.g., a human patient in need of such a therapy, a therapeutically effective amount of annamycin or a pharmaceutically acceptable liposomal formulation or prodrug thereof and an anticancer compound. In one embodiment, the anticancer compound is trabectedin. In a particular embodiment, the method of the present invention involves the administration to a mammal, e.g., a human patient, in need of such a therapy a therapeutically effective amount of annamycin or a pharmaceutically acceptable liposomal formulation or prodrug thereof and a therapeutically effective amount of trabectedin or a pharmaceutically acceptable salt or liposomal formulation thereof to treat pancreatic cancer. In one embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of trabectedin or a pharmaceutically acceptable salt or liposomal formulation thereof to treat an adenocarcinoma. In one embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of trabectedin or a pharmaceutically acceptable salt or liposomal formulation thereof to treat pancreatic ductal adenocarcinoma.

In one embodiment, the method of the present involves the administration to a mammal, e.g., a human patient in need of such a therapy, a therapeutically effective amount of annamycin or a pharmaceutically acceptable liposomal formulation or prodrug thereof and an anticancer compound. In one embodiment, the anticancer compound is ifosfamide. In a particular embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of ifosfamide or a pharmaceutically acceptable salt or liposomal formulation thereof to treat a sarcoma. In one embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of ifosfamide or a pharmaceutically acceptable salt or liposomal formulation thereof to treat osteosarcoma.

In one embodiment, the method of the present involves the administration to a mammal, e.g., a human patient in need of such a therapy, a therapeutically effective amount of annamycin or a pharmaceutically acceptable liposomal formulation or prodrug thereof and an anticancer compound. In one embodiment, the anticancer compound is cyclophosphamide. In a particular embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of cyclophosphamide or a pharmaceutically acceptable salt or liposomal formulation thereof to treat diseases including but not limited to: mesothelioma, bladder cancer, lung cancer, pancreatic cancer, skin cancer, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma, cancer of the urethra, cancer of the penis, testicular cancer, hepatocellular (hepatic and/or biliary duct) cancer, primary or secondary central nervous system tumor, primary or secondary brain tumor, osteosarcoma, Hodgkin's disease, chronic or acute leukemia, acute myeloid leukemia, acute lymphocytic leukemia (ALL), chronic myeloid leukemia, lymphocytic lymphoma, lymphoblastic leukemia, follicular lymphoma, lymphoid malignancies of T-cell or B-cell origin, melanoma, multiple myeloma, oral cancer, prostate cancer, cancer of the kidney and/or meter, renal cell carcinoma, carcinoma of the renal pelvis, neoplasm of the central nervous system, primary central nervous system lymphoma, non-Hodgkin's lymphoma, spinal axis tumor, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In one embodiment, the method of the present involves the administration to a mammal, e.g., a human patient in need of such a therapy, a therapeutically effective amount of annamycin or a pharmaceutically acceptable liposomal formulation or prodrug thereof and an anticancer compound. In one embodiment, the anticancer compound is irinotecan. In a particular embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of irinotecan or a pharmaceutically acceptable salt or liposomal formulation thereof to treat a pancreatic cancer. In one embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of irinotecan or a pharmaceutically acceptable salt or liposomal formulation thereof to treat an adenocarcinoma. In one embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of irinotecan or a pharmaceutically acceptable salt or liposomal formulation thereof to treat pancreatic ductal adenocarcinoma.

In one embodiment, the method of the present involves the administration to a mammal, e.g., a human patient in need of such a therapy, a therapeutically effective amount of annamycin or a pharmaceutically acceptable liposomal formulation or prodrug thereof and an anticancer compound. In one embodiment, the anticancer compound is gemcitabine. In a particular embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of gemcitabine or a pharmaceutically acceptable salt or liposomal formulation thereof to treat a pancreatic cancer. In one embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of gemcitabine or a pharmaceutically acceptable salt or liposomal formulation thereof to treat an adenocarcinoma. In one embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of gemcitabine or a pharmaceutically acceptable salt or liposomal formulation thereof to treat pancreatic ductal adenocarcinoma.

In one embodiment, the method of the present involves the administration to a mammal, e.g., a human patient in need of such a therapy, a therapeutically effective amount of annamycin or a pharmaceutically acceptable liposomal formulation or prodrug thereof and an anticancer compound. In one embodiment, the anticancer compound is a KRAS inhibitor. In a particular embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of a KRAS inhibitor or a pharmaceutically acceptable salt or liposomal formulation thereof to treat a pancreatic cancer. In one embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of a KRAS inhibitor or a pharmaceutically acceptable salt or liposomal formulation thereof to treat an adenocarcinoma. In one embodiment, the method of the present invention involves the administration to a mammal, e.g., a human patient, in need of such a therapy, a therapeutically effective amount of annamycin or a pharmaceutically acceptable liposomal formulation or prodrug thereof and a therapeutically effective amount of a KRAS inhibitor or a pharmaceutically acceptable salt or liposomal formulation thereof to treat pancreatic ductal adenocarcinoma.

### VII. Combination Treatments

In addition to the combination therapies and pharmaceutical products described herein, certain embodiments of the present disclosure provide for the administration or application of one or more additional forms of therapies for the treatment or prevention of a disease. For example, the disease may be a hyperproliferative disease, such as cancer.

The additional form of therapy may be administration of one or more additional pharmacological agents that can be applied in the treatment or prevention of cancer. If the additional therapy is a pharmacological agent, it may be administered prior to, concurrently, or following administration of the combination therapy and/or pharmaceutical product described herein.

The interval between the administration of the combination therapy and/or pharmaceutical product described herein and the additional therapy may be any interval as determined by those of ordinary skill in the art. For example, the interval may be minutes to weeks. In embodiments where the agents are separately administered, one would generally ensure that a long period of time did not expire between the time of each delivery, such that each therapeutic agent would still be able to exert an advantageously combined effect on the subject. For example, the interval between therapeutic agents may be about 12 h to about 24 h of each other and, more preferably, within about 6 hours to about 12 h of each other. In some situations, the time period for treatment may be extended, however, where several days (2, 3, 4, 5, 6 or 7) to several weeks (1, 2, 3, 4, 5, 6, 7 or 8) lapse between the respective administrations. In some embodiments, the timing of administration of an additional therapeutic agent is determined based on the response of the subject to the nanoparticles.

Various combinations may be employed. For the example below a pharmaceutical product described herein is “A” and an additional therapy is “B”:

### A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of any compound or therapy of the present disclosure to a patient will follow general protocols for the administration of such compounds, taking into account the toxicity, if any, of the agents. Therefore, in some embodiments there is a step of monitoring toxicity that is attributable to combination therapy. It is expected that the treatment cycles may be repeated. It also is contemplated that various standard therapies, as well as surgical intervention, may be applied in combination with the described therapy.

In specific aspects, it is contemplated that a standard therapy will include chemotherapy, radiotherapy, immunotherapy, surgical therapy or gene therapy and may be employed in combination with the inhibitor of gene expression therapy, anticancer therapy, or both the inhibitor of gene expression therapy and the anti-cancer therapy, as described herein.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Non-limiting examples of chemotherapeutic agents include alkylating agents, such as thiotepa, and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; Bcl-2 inhibitors such as oblimersen, ABT-737, navitoclax, venetoclax, sonrotoclax, and lisaftoclax; antibiotics, such as the enediyne antibiotics (e.g., calicheamicin, especially calicheamicin gammalI and calicheamicin omegal1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dronanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; KRAS inhibitors such as sotorasib, adagrasib, divarasib, D3S-001, BI 3706674, and PF-07934040; platinum; etoposide (VP-16); ifosfamide; mitoxantrone; vincristine; vinorelbine; novantrone; trabectedin; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

### 2. Radiotherapy

Other factors that cause DNA damage and have been used extensively include what are known as γ-rays, X-rays, and/or the directed delivery of radioisotopes to tumor cells. Other forms of DNA damaging factors are also contemplated, such as microwaves, proton beam irradiation (U.S. Patent No. 5,760,395 and 4,870,287), and UV-irradiation. It is likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, may rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment. As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell may bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds; cytokine therapy, e.g., interferons IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication No. WO2015016718; incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156, can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. Further examples can therefore be contemplated. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VIII. Examples

The following examples are included to demonstrate preferred embodiments of the invention. It should be appreciated by those of skill in the art that the techniques disclosed in the example which follows represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Combination Efficacy of Liposomal Formulation of Annamycin (L-ANN) and Alkylating Agents (Ifosfomide, IFO) in Experimental Model of Lung Metastasis Od Fibrosarcoma Origin

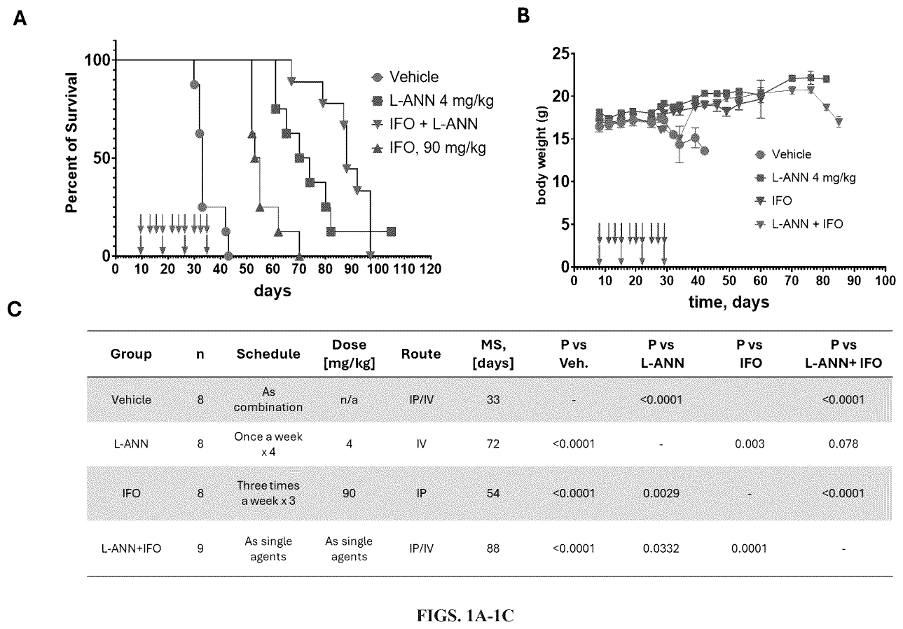

The objective of this experiment was to assess the combination benefits of ANN and IFO treatment in experimental lung metastasis model of fibrosarcoma. Female C57B6 mice were injected intravenously with 1×10⁵ mouse MCA205 fibrosarcoma cells. On day 10 animals were randomized into four experimental groups (n=8-10) receiving (i) vehicle (n=8), L-ANN at 4 mg/kg (n=8), IFO at 90 mg/kg (n=8) and combination of both. Kaplan-Meyer analysis showed clear extension of the survival in mice treated with IFO and L-ANN alone with median survival (MS) of 54 and 72 days respectively. MS of vehicle-treated mice was 33 days. Combination of L-ANN and IFO lead to significant extension of the survival when compared to single agent-treated mice with p=0.0029 for IFO and 0.042 for L-ANN respectively (FIG. 1A, FIG. 1C). The treatment was well tolerated by mice with no significant body weight change in single agent treated mice. The combination led to significant drop in body weight, however mice completely recovered after completion of the treatment (FIG. 1B). Combined treatment of L-ANN and IFO led to significant extension of the survival in experimental lung metastatic model.

### Example 2: Combination of Efficacy of Liposomal Formulation of Annamycin (L-ANN) and Topoisomerase I Poison (Irinotecan) in Orthotopic Model of Pancreatic Ductal Adenocarcinoma

The objective of this study was to assess the combination benefits of topoisomerase I (Irinotecan, IRINO) and II (L-ANN) poisons in orthotopic model of pancreatic ductal adenocarcinoma (PDAC). Female nude athymic mice were surgically implanted with 1 million of human MDA PATC53 Luc PDAC cells (intrapancreatic tumor injection). On the day 8th, the animals were randomized into four experimental groups receiving i) vehicle, ii) irinotecan (IRINO), 5 mg/kg, iii) L-ANN 4 mg/kg (n=10) and iv) combination of both (n=9). The rate of tumor growth was monitored using bioluminescent imaging (BLI), 10 minutes after subcutaneous administration of D-Luciferin at 75 mg/kg. Combination benefits were assessed using Kaplan-Mayer survival analysis and compared between the vehicle-and single-agent treated groups.

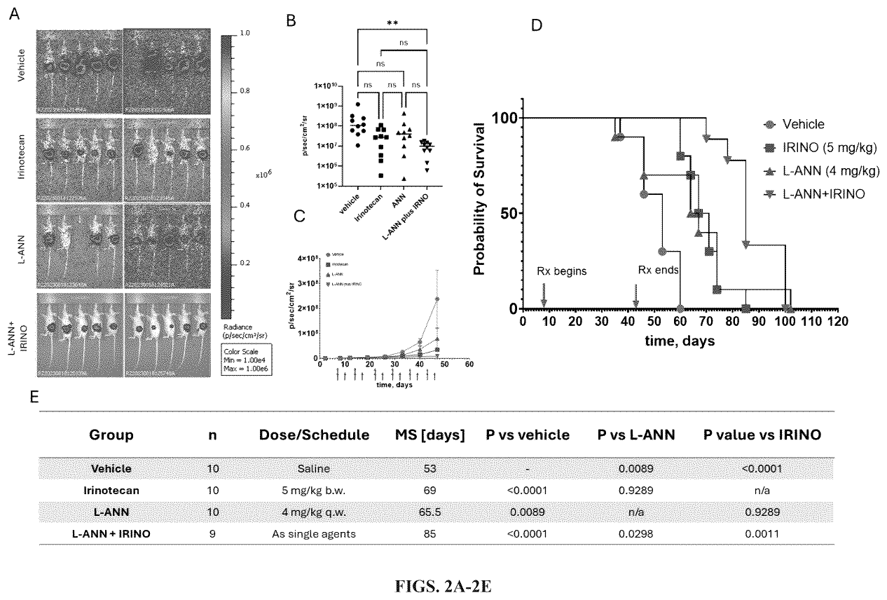

Bioluminescent imaging confirmed tumor take across all experimental groups with no statistical difference in intensities between the groups. Following the treatment, there was a clear separation of the intensities between experimental groups. FIG. 2 is an example of bioluminescent signal distribution at day 47 following tumor implantation (FIGS. 2A and 2B), along with longitudinal analysis (FIG. 2C). Based on BLI, both topoisomerase poisons reduced tumor growth when compared to vehicle-treated cohort, however they failed to reach the statistical significance by day 47 of the study. In contrast, combined treatment led to significant inhibition of tumor growth vs vehicle (p=0.0011), but not vs single agent-treated groups (FIG. 2B). Survival analysis confirmed statistical extension of the survival for combination group when compared to the vehicle (p<0.0001), but also to single agent-treated mice with p=0.0298 for L-ANN and 0.0011 for IRINO. The corresponding MS was 53 days for vehicle, 69 for IRINO, 65.5 for L-ANN and 85 days for combination group (FIG. 2E). Combined treatment of L-ANN and IRINO resulted in significant extension of the survival in human orthotopic pancreatic ductal adenocarcinoma models.

### Example 3: Combination of Efficacy of Liposomal Formulation of Annamycin (L-ANN) and Gemcitabine (GEM) in Orthotopic Model of Pancreatic Ductal Adenocarcinoma

The objective of this study was to assess the combination benefits of Gemcitabine (Gem, and L-ANN in human orthotopic model of pancreatic ductal adenocarcinoma (PDAC). Female nude athymic mice were orthotopically implanted with 1.0 million of human MDA PATC53 PDAC cells in mixture with Matrigel (50%, 50 μl). On day 8, the animals were randomized into four experimental groups (n=10) receiving i) vehicle, ii) gemcitabine 50 mg/kg, iii) L-ANN at 2 mg/kg and iv) combination of gemcitabine and L-ANN. Both agents were administered on a once-a-week schedule for six cycles. Survival was the primary output of the study.

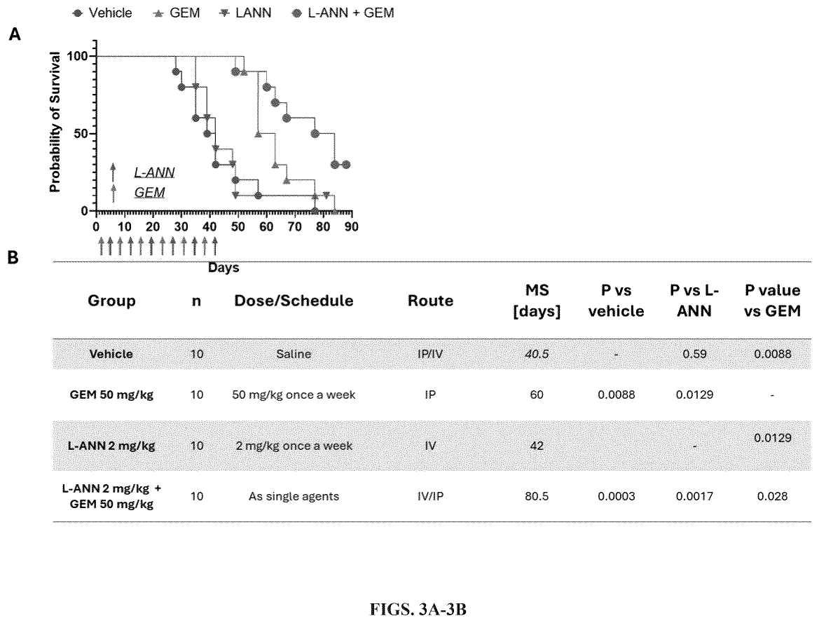

The mice treated with the combination regimen (L-ANN plus GEM) showed the median survival of 80.5 days, followed by GEM-only group (60 days) and L-ANN 42 days. The vehicle-treated group has a MS value of 40.5 days (FIGS. 3A-C). The treatment was well tolerated, and no significant body weight reduction was observed either in single-agent or combination group (FIG. 3B). Combined treatment of L-ANN and GEM resulted in significant extension of the survival in human orthotopic pancreatic ductal adenocarcinoma models.

### Example 4: Efficacy of Combination of Liposomal Formulation of Annamycin (L-ANN) and Vincristine (VCR) in Mouse Acute Myeloid Leukemia Model

The objective of this study was to assess the combination benefits of vincristine (VCR), and L-ANN in in mouse model of mouse acute myeloid leukemia. Female C57B6 albino mice received bolus, IV injection of AML-Turq-2 cells. Day five following tumor inoculation, the mice were randomized into four experimental groups (n=5) receiving i) vehicle, ii) L-ANN 4 mg/kg, iii) VCR 1 mg/kg and iv) combination of L-ANN and VCR.

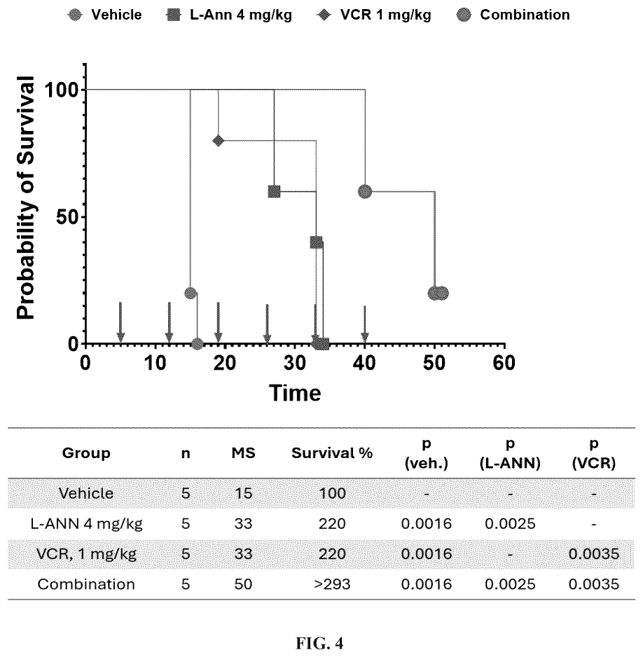

Mice that received AML-Turq2 model and treated with vehicle progressed rapidly and had to be euthanized due to acute onset of blast crisis observed approximately two weeks after injection (FIG. 4). Single agents treated animals showed significant extension of the survival when compared to vehicle treated animal (33 days for ANN and VCR vs 15 days for the vehicle). Mice receiving Combination of VCR and L-ANN at 1 and 4 mg/kg showed further increase of the survival with MS value of 50 days (p=0.0016 vs vehicle, p=0.0025 for L-ANN and p=0.0035 for VCR). Kaplan-Meyer survival plots along with statistical analysis have been presented on FIG. 4. Statistically significant extension of survival for single-agent treated mice (median survival 15 vs 33 days, p=0.0016). Combined treatment led to further extension of the survival. The difference in survival of mice receiving combination was statistically significant when compared to vehicle (p=0.0016), but also single agent treated mice (p=0.0035 and 0.0025).

### Example 5: Efficacy of Combination of Liposomal Formulation of Annamycin (L-ANN) and Ifosfamide (IFO) in Human Osteosarcoma Model

The objective of this experiment was to assess the combination benefits of ANN and IFO treatment in experimental lung metastasis model of osteosarcoma fibrosarcoma. Human osteosarcoma cells (143B) were injected subcutaneously into female athymic mice. On day ten when the average tumor volume reached ~100 mm³, the mice were randomized into four groups (n=10) receiving i) vehicle, ii) IFO 90 mg/kg, L-ANN 3 mg/kg and iv) combination of both. L-ANN was administered once a week and IFO was given on a three-times-a-week schedule (Mon-Wed-Fri). The tumor growth was monitored based on tumor volume, estimated using ellipsoid equation based on tumor diameter measurement.

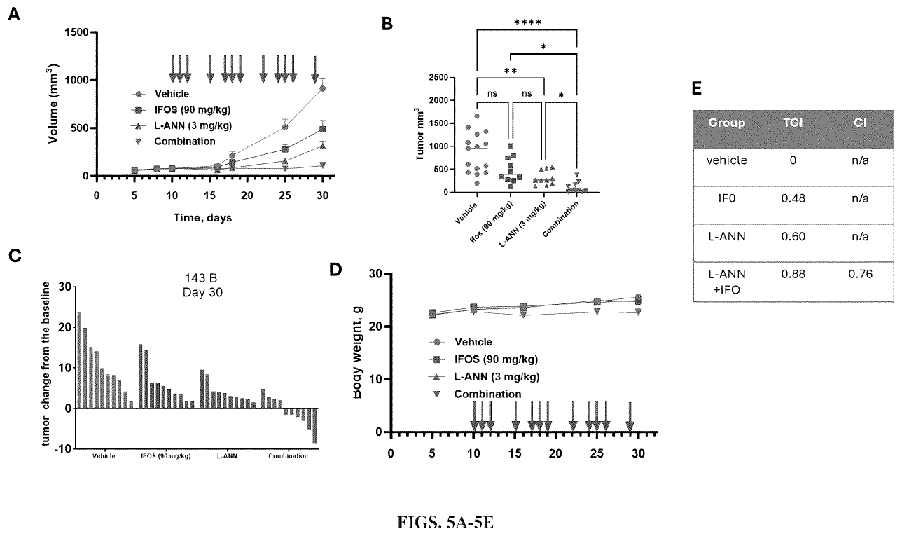

143B cells form an aggressive tumor reaching an average of 1,000 mm³ in about one month after implantation in vehicle treated mice (FIG. 5A). Tumor volume distribution analysis at the study termination, confirmed L-ANN-induced inhibition (p=0.0019), but did not reach the significance for IFO (p=0.099). Significant reduction of tumor growth was however achieved in the combination regimen with p=0.0002 at the study termination (FIG. 5B). Importantly, combination led to statistically significant inhibition also when compared to the single agent-treated groups with p=0.012 for IFO and p=0.022 for L-ANN. Six out of ten mice in this group exhibited reduction of the tumor growth from the baseline (negative values on the plot, FIG. 5C). There was no significant body weight decrease across all tested groups throughout the study (FIG. 5D). FIG. 5E presents tumor growth inhibition values (TGI) computed for each experimental group along with combination index CI for the L-ANN plus IFO-treated group. CI values equal 1.0 indicate an additive effect, below 1.0 shows synergy and antagonistic effect is defined for C is greater than 1. The combination of L-ANN and IFO shows improved efficacy when compared to single-agent treated regimens in subcutaneous human osteosarcoma model.

### Example 6: In Vitro Evaluation of Combination Efficacy of Trabectedin (TRB) and Annamycin (ANN) in Pancreatic Ductal Adenocarcinoma (PDAC)

The objective of this experiment was to assess in vitro the combination benefits of ANN and Trabectedin in PDAC model. ATTC available Mpanc-96 cells were plated in 96 well plate at 3×10∧3 cells/well and incubated overnight at 5% CO2 at 37C. The next day, the cells were exposed to increasing concentration of ANN and Trabectedin in stable ratio 50:1 and constant vehicle (0.5% DMSO). After 72 hours, the viability of the cells was assessed using Resazurin assay. Viability was normalized to vehicle and “no cell”-treated control, and expressed as percentage, The viability matrix was analyzed using Bliss algorithm in Combenefit Software to identify potential region of synergy.

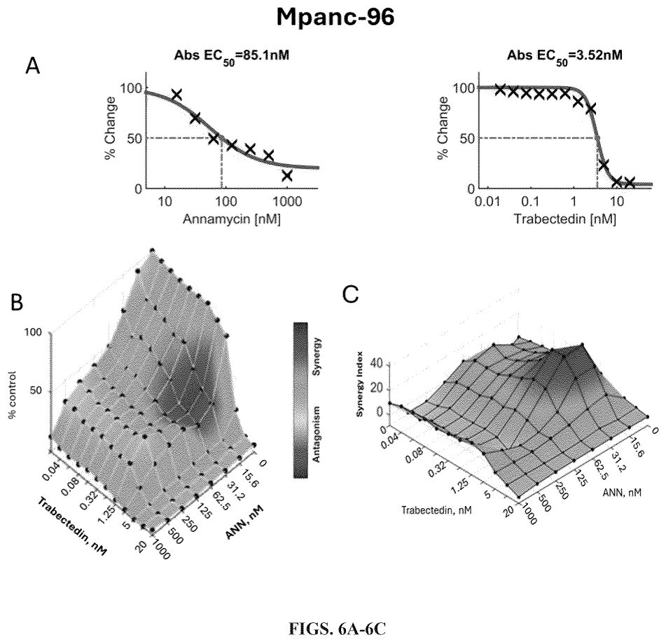

Both Trabectedin and Annamycin showed dose-dependent cytotoxic activity when applied as single agent with IC50 of 85.1 and 3.52 nM for ANN and TRB respectively (FIG. 6A). Combination of both agents with variable ratio of ANN to TRB showed region of strong synergy manifested in high Bliss Synergy index (Blue area, FIG. 6B). For example, cells treated with ANN at 31.25 nM and TRB at 1.25 nM showed increase of Bliss Index to 43-44, indicating strong synergy in this concentration range (FIG. 6C).

### REFERENCES

The following references, to the extent that they provide exemplary procedural or other details supplementary to those set forth herein, are specifically incorporated herein by reference.

Anderson, Practical Process Research & Development—A Guide for Organic Chemists, 2nd ed., Academic Press, New York, 2012.

Handbook of Pharmaceutical Salts: Properties, and Use, Stahl and Wermuth Eds., Verlag Helvetica Chimica Acta, 2002.

Reagan-Shaw et al., FASEB J., 22(3):659-661, 2008.

Smith, March's Advanced Organic Chemistry: Reactions, Mechanisms, and Structure, 7th Ed., Wiley, 2013.

What is claimed is:

1. A method of treating a cancer, the method comprising administering to a patient in need thereof a therapeutically effective amount of annamycin or a pharmaceutically acceptable liposomal formulation or prodrug thereof in combination with a therapeutically effective amount of a second anticancer compound or a pharmaceutically acceptable salt or liposomal formulation, or prodrug thereof.

2. The method of claim 1, wherein the second anticancer compound is an alkylating agent.

3. The method of claim 1, wherein the second anticancer compound is cytarabine, azacitidine, venetoclax, vincristine, trabectedin, ifosfamide, cyclophosphamide, irinotecan, or gemcitabine.

4. The method of claim 1, wherein the second anticancer compound is a KRAS inhibitor.

5. The method of claim 4, wherein the second anticancer compound is a pan-KRAS inhibitor.

6. The method of claim 4, wherein the second anticancer compound is a selective KRAS inhibitor.

7. The method according to any one of claims 1-6, wherein the therapeutic effect in the patient is greater than the aggregate achieved by either annamycin or the second anticancer compound.

8. The method according to any one of claims 1-7, wherein the cancer is metastatic.

9. The method according to any one of claims 1-8, wherein the cancer is drug-resistant.

10. The method according to any one of claims 1-9, wherein the cancer is selected from the group consisting of mesothelioma, bladder cancer, lung cancer, pancreatic cancer, skin cancer, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma, cancer of the urethra, cancer of the penis, testicular cancer, hepatocellular (hepatic and/or biliary duct) cancer, primary or secondary central nervous system tumor, primary or secondary brain tumor, osteosarcoma, Hodgkin's disease, chronic or acute leukemia, acute myeloid leukemia, acute lymphocytic leukemia (ALL), chronic myeloid leukemia, lymphocytic lymphoma, lymphoblastic leukemia, follicular lymphoma, lymphoid malignancies of T-cell or B-cell origin, melanoma, multiple myeloma, oral cancer, prostate cancer, cancer of the kidney and/or ureter, renal cell carcinoma, carcinoma of the renal pelvis, neoplasm of the central nervous system, primary central nervous system lymphoma, non-Hodgkin's lymphoma, spinal axis tumor, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

11. The method of claim 10, wherein the cancer is sarcoma.

12. The method of claim 10, wherein the cancer is pancreatic cancer.

13. The method of claim 12, wherein the cancer is pancreatic ductal adenocarcinoma.

14. The method of claim 10, wherein the cancer is acute myeloid leukemia or acute lymphocytic leukemia.

15. The method of claim 10, wherein the cancer is lung cancer.

16. The method of claim 15, wherein the lung cancer is lung metastasis of fibrosarcoma origin.

17. The method of claim 10, wherein the cancer is osteosarcoma.

18. The method according to claims 1-17, wherein the patient is a mammal.

19. The method of claim 18, wherein the patient is a human.

20. The method according to any one of claims 1-19, wherein annamycin is administered concurrently with the second anticancer compound.

21. The method according to any one of claims 1-19, wherein annamycin is administered sequentially with the second anticancer compound.

22. The method according to any one of claims 1-20, wherein annamycin and the second anticancer compound are co-formulated.

23. The method of claim 22, wherein annamycin and the second anticancer compound are co-formulated in a pharmaceutical composition comprising a pharmaceutically acceptable excipient.

24. The method according to any one of claims 1-19, wherein annamycin and the second anticancer compound are formulated in separate dosage forms.

25. The method according to any one of claims 1-24, wherein the method further comprises administering a pharmaceutically effective amount of at least one immunotherapeutic agent to the patient.

26. The method according to any one of claims 1-25, wherein the method further comprises administering radiation therapy to the patient.

27. The method according to any one of claims 1-26, wherein treating a cancer in a patient in need thereof comprises one or more of the following:
(a) increasing survival time of the patient;
(b) reducing volume of the primary cancer;
(c) retarding growth of the primary cancer;
(d) reducing the number of metastatic tumors;
(e) reducing the volume of metastatic tumors; and
(f) retarding growth of metastatic tumors.

28. The method according to any one of claims 1-27, wherein the cancer is a solid tumor and the treatment results in a reduction in tumor size of at least 15%.

29. The method of claim 28, wherein the reduction in tumor size is at least 30%.

30. The method according to any one of claims 1-29, wherein treating cancer does not result in an annamycin-induced toxicity of such severity that repeated administration of annamycin is contraindicated.

31. The method of claim 30, wherein the annamycin-induced toxicity is cardiotoxicity.

32. A pharmaceutical product comprising:
(a) a first composition comprising annamycin; and
(b) a second composition comprising cytarabine, azacitidine, venetoclax, vincristine, trabectedin, ifosfamide, cyclophosphamide, irinotecan, gemcitabine, or a KRAS inhibitor.

33. The pharmaceutical product of claim 32, wherein the first composition further comprises a pharmaceutically acceptable excipient.

34. The pharmaceutical product of claim 32, wherein the second composition further comprises a pharmaceutically acceptable excipient.

35. The pharmaceutical product according to any one of claims 32-34, wherein the pharmaceutical product is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

36. The pharmaceutical product of claim 35, wherein the pharmaceutical product is formulated for oral administration.

37. The pharmaceutical product of claim 35, wherein the pharmaceutical product is formulated for administration via injection.

38. A method of treating cancer in a patient in need thereof, comprising administering to the patient pharmaceutical product according to any one of claims 32-37.
